(19) United States
(12) Patent Application Publication
(10) Pub. No.: US 2021/0308080 A1
(43) Pub. Date: Oct. 7, 2021

(54) TRANSIENT INDUCED SYSTEMIC ACIDOSIS (TISA) TO TREAT VIRUS INFECTIONS

(71) Applicants: Sarfaraz K. Niazi, Deerfield, IL (US); Sunitha Lokesh, Rolling Meadows, IL (US)

(72) Inventors: Sarfaraz K. Niazi, Deerfield, IL (US); Sunitha Lokesh, Rolling Meadows, IL (US)

(21) Appl. No.: 16/837,861

(22) Filed: Apr. 1, 2020

(51) Int. Cl. A61K 31/19; A61K 33/04; A61K 33/42; A61K 33/20

(52) U.S. Cl. CPC A61K 31/19; A61K 33/20; A61K 33/42; A61K 33/04

(57) ABSTRACT

Transient induced systemic acidosis (TISA) is a universal treatment of all virus infections by repeatedly inducing acidosis of plasma for brief periods to inactivate the virus in the body, without any deleterious effects of acidosis because of its transient nature.

### BACKGROUND OF THE INVENTION

A virus is a small infectious organism—much smaller than a fungus or bacterium—that must invade a living cell to reproduce (replicate). The virus attaches to a cell (called the host cell), enters the cell, and releases its DNA or RNA inside the cell. The virus's DNA or RNA is the genetic material containing the information needed to make copies of (replicate) the virus. The virus's genetic material takes control of the cell and forces it to replicate the virus. The infected cell usually dies because the virus keeps it from performing its normal functions. When it dies, the cell releases new viruses, which go on to infect other cells.

Viruses are classified as DNA viruses or RNA viruses, depending on whether they use DNA or RNA to replicate. RNA viruses include retroviruses, such as HIV (human immunodeficiency virus). RNA viruses, particularly retroviruses, are prone to mutate.

Some viruses do not kill the cells they infect but instead alter the cell's functions. Sometimes the infected cell loses control over normal cell division and becomes cancerous.

Some viruses, such as hepatitis B virus and hepatitis C virus, can cause chronic infections. Chronic hepatitis can last for years, even decades. In many people, chronic hepatitis is quite mild and causes little liver damage. However, in some people, it eventually results in cirrhosis (severe scarring of the liver), liver failure, and sometimes liver cancer.

A virus takes control of the cell it infects and forces it to make more viruses.

Viruses usually infect one particular type of cell. For example, common cold viruses infect only cells of the upper respiratory tract. Additionally, most viruses infect only a few species of plants or animals. Some infect only humans.

The most common respiratory infections are upper respiratory infections, which include sore throat, sinusitis, and the common cold; other viral respiratory infections include influenza and pneumonia. In small children, viruses also commonly cause croup (which is inflammation of the upper and lower airways, called laryngotracheobronchitis) or lower airways (bronchiolitis). Respiratory infections are more likely to cause severe symptoms in infants, older people, and people with lung or heart disorders.

Other viruses infect other specific parts of the body, such as:
Gastrointestinal tract: Infections of the gastrointestinal tract, such as gastroenteritis, are commonly caused by viruses, such as noroviruses and rotaviruses;
Liver: These infections result in hepatitis;
Nervous system: Some viruses, such as the rabies virus and the West Nile virus, infect the brain, causing encephalitis.
Brain: Infecting the layers of tissue that cover the brain and spinal cord (meninges), causing meningitis or polio.
Skin: Viral infections that affect only the skin sometimes result in warts or other blemishes. Many viruses that affect other parts of the body, such as chickenpox, also cause a rash;
Placenta and fetus: Some viruses, such as the Zika virus, the rubella virus, and cytomegalovirus, can infect the placenta and fetus in pregnant women.

There are some viruses that can also affect multiple body systems. Such viruses include enteroviruses (such as coxsackieviruses and echoviruses) and cytomegaloviruses.

Viruses are spread (transmitted) in various ways, including direct contact, indirect contact, droplet contact, airborne, and via a common vehicle (e.g., food, water, medical devices, etc.). They may be swallowed or inhaled, spread by the bites of insects, such as mosquitoes, certain biting flies, or ticks, read sexually (in sexually transmitted diseases), spread during transfusion of contaminated blood.

Many viruses that were once present in only a few parts of the world are now spreading. These viruses include chikungunya virus, Crimean-Congo hemorrhagic fever virus, Japanese encephalitis virus, Rift Valley Fever virus, West Nile virus, Avian influenza (H5N1), Ross River virus, Ebola virus, Zika virus, and louping ill virus, and most recently the coronavirus responsible for COVID-19. These viruses are spreading partly because climate change has resulted in more vector-borne (e.g., mosquitoes) diseases. Also, travelers may be infected, then return home and be bitten by a mosquito, which spreads the virus to other people.

The body has several defenses against viruses: physical barriers, such as the skin, which discourage easy entry, the body's immune defenses, which attack the virus. When a virus enters the body, it triggers the body's immune defenses. These defenses begin with white blood cells, such as lymphocytes and monocytes, which learn to attack and destroy the virus or the cells the virus has infected. If the body survives the virus attack, some of the white blood cells remember the invader and can respond more quickly and effectively to subsequent infection by the same virus. This response is called immunity. Immunity is also produced by getting a vaccine.

Some viruses alter the DNA of their host cells in a way that helps cancer develop. Some viruses, such as herpesviruses and HIV, leave their genetic material in the host cell, where the material remains dormant for an extended time (called latent infection). When the cell is disturbed, the virus may begin replicating again and cause disease. Only a few viruses are known to cause cancer, but there may be others.

Common viral infections (such as measles, rubella, or chickenpox) may be diagnosed based on symptoms. For infections that occur in epidemics (such as influenza), the presence of other similar cases may help doctors identify a particular infection. For other infections, blood tests and cultures (growing microorganisms in the laboratory from samples of blood, body fluid, or other material taken from an infected area) may be done. Polymerase chain reaction (PCR) techniques are used to make many copies of the viral genetic material. PCR techniques make it easier for doctors to rapidly and accurately identify the virus. Blood is also tested for antigens, which are proteins on or in viruses that trigger the body's defense. Blood is also tested for antibodies to viruses. Antibodies are proteins produced by the immune system to help defend the body against a particular attack. Tests are done quickly, especially when the infection is a serious threat to public health or when symptoms are severe.

Vaccines and immune globulins help the body better defend itself against diseases caused by certain viruses (or bacteria). The process of strengthening the body's defenses is called immunization. People can help prevent many viral infections by commonsense measures to protect themselves and others (personal protective measures). These measures vary depending on how the virus spreads. Measures include the following: frequently and thoroughly washing the hands with soap, Consuming only food and liquids that are appropriately prepared or treated, avoiding contact with infected people and contaminated surfaces

Sneezing and coughing into tissues or into the upper arm, completely covering the mouth and nose, using safe-sex practices, preventing bites by ticks, mosquitoes, and other arthropods.

Vaccines work by stimulating the body's natural defense mechanisms (called active immunization). Vaccines are given before exposure to a virus to prevent infection.

Viral vaccines in general use include the following: Hepatitis A, Hepatitis B, Human papillomavirus (HPV), Influenza, Japanese encephalitis (inflammation of the brain),

Measles, mumps, and rubella, Polio, Rabies, Rotavirus, Varicella, Shingles (herpes zoster), Yellow fever. A smallpox vaccine is available but is used only in people who are at high risk of getting the infection, such as certain military personnel.

Viral diseases can be eradicated by good vaccines. Smallpox was eradicated in 1978. Polio has been eradicated from all but a few countries where logistics and religious sentiment continue to interfere with vaccination. Measles has been almost eradicated from some parts of the world, such as the Americas. However, because measles is highly contagious and vaccination coverage is incomplete even in regions where it is considered eradicated, it is not likely to be completely eradicated soon.

Immune globulins are a sterilized solution of antibodies (also called immunoglobulins) collected from the blood of a group of people. Immune globulins are given directly to a person (called passive immunization). Immunoglobulins can be collected from the blood of the following: people who are generally healthy (these immunoglobulins are called pooled human immunoglobulin). These people have many antibodies that defend against a specific infectious organism; often, because they have been infected with that organism (these immunoglobulins are called hyperimmune globulin), hyperimmune globulin is available for only a few infectious diseases, such as hepatitis B, rabies, tetanus, and chickenpox.

Immune globulins are given by injection into a muscle or a vein. The immunity provided by immune globulins lasts for only a few days or weeks until the body eliminates the injected antibodies. Some immune globulins and some vaccines, such as those for rabies and hepatitis B, are also used after exposure to the virus to help prevent infection from developing or reduce the severity of the infection. Immune globulins may also help treat some infections. For example, they may be given to people whose immune system does not respond adequately to infection.

There are no specific treatments for many viruses. Drugs that combat viral infections are called antiviral drugs. There are no effective antiviral drugs for many viral infections. However, there are several drugs for influenza, many drugs for infection by one or more herpesviruses (see Table: Some Antiviral Drugs for Herpesvirus Infections), and many new antiviral drugs for the treatment of HIV and hepatitis C. Many antiviral drugs work by interfering with the replication of viruses. Most drugs used to treat HIV infection work this way.

Because viruses are tiny and replicate inside cells using the cells' metabolic functions, there are only a limited number of metabolic functions that antiviral drugs can target. In contrast, bacteria are relatively large organisms, commonly reproduce by themselves outside of cells, and have many metabolic functions that antibacterial drugs (antibiotics) can target. Therefore, antiviral drugs are much more difficult to develop than antibiotics. Also, unlike antibiotics, which are usually effective against many different species of bacteria, most antiviral drugs are usually effective against only one (or a very few) viruses.

Antiviral drugs can be toxic to human cells. Also, viruses can develop resistance to antiviral drugs. Most antiviral drugs can be given by mouth. Some can also be given by injection into a vein (intravenously) or muscle (intramuscularly). Some are applied as ointments, creams, or eye drops or are inhaled as a powder. Antibiotics are not effective against viral infections, but if a person has a bacterial infection in addition to a viral infection, an antibiotic is often necessary.

Interferon drugs are replicas of naturally occurring substances that slow or stop viral replication. These drugs treat certain viral infections such as chronic hepatitis B, chronic hepatitis C, genital warts.

### BRIEF DESCRIPTION OF THE INVENTION

The economic cost of managing and treating virus infections runs into billions of dollars, in addition to lost human resources due to these infections. There is a dire need to develop a treatment that directly acts to inactivate the virus in the body, without any specific activity of the treatment modality. Given that viruses, like all other microorganisms, can be deactivated by applying physical or chemical exposure. For example, ultraviolet light, exposure to alcohol, such as in hand sanitizers and other chemical exposures, can be effective in inactivating the virus. However, none of these techniques are applied to the virus load that has already begun residing in the body, where it runs its course before being inactivated by the body's immune system.

The present invention applies a novel approach to inactivating viruses by altering the pH of plasma momentarily and periodically to gradually inactivate the virus load, leading to the elimination of all viruses from the body. Human blood contains a buffer of carbonic acid and bicarbonate anion to maintain blood pH between 7.35 and 7.45, as a value higher than 7.8 or lower than 6.8 can lead to death. In this buffer, hydronium and bicarbonate anion are in equilibrium with carbonic acid. Furthermore, the carbonic acid in the first equilibrium can decompose into carbon dioxide gas and water, resulting in a second equilibrium system between carbonic acid and water. Because carbon dioxide is an essential component of the blood buffer, its regulation in the body, as well as that of oxygen, is essential. The effect of this can be significant when the human body is subjected to strenuous conditions. In the body, there exists another equilibrium between hydronium and oxygen, which involves the binding ability of hemoglobin. An increase in hydronium causes this equilibrium to shift towards the oxygen side, thus releasing oxygen from hemoglobin molecules into the surrounding tissues/cells. This system continues during exercise, providing continuous oxygen to working tissues.

Acidosis is a process causing increased acidity in the blood and other body tissues (i.e., an increased hydrogen ion concentration). If not further qualified, it usually refers to the acidity of the plasma. The term acidemia describes the state of low blood pH, while acidosis is used to describe the processes leading to these states. Nevertheless, the terms are sometimes used interchangeably.

The distinction may be relevant where a patient has factors causing both acidosis and alkalosis, wherein the relative severity of both determines whether the result is a high, low, or normal pH. Acidemia is said to occur when arterial pH falls below 7.35, while its counterpart (alkalemia) occurs at a pH of over 7.45. Arterial blood gas analysis and other tests are required to separate the leading causes. The rate of cellular metabolic activity affects and, at the same time, is affected by the pH of the body fluids. In mammals, the normal pH of arterial blood lies between 7.35 and 7.50 depending on the species (e.g., healthy human-arterial blood pH varies between 7.35 and 7.45). Blood pH values compatible with life in mammals are limited to a pH range between 6.8 and 7.8. Changes in the pH of arterial blood (and therefore the extracellular fluid) outside this range result in irreversible cell damage.

The present invention is based on the sensitivity of the viruses to acidic pH and a method of reducing the pH of plasma for a short time, avoid damage to the body, yet long enough to cause a breakdown of viruses; repeating the cycle results in significant inactivation of the virus, often to a level where the body can itself take care of removing the rest. This approach is particularly useful for enveloped viruses that are sensitive to smaller pH changes than the non-enveloped viruses, yet, all viruses undergo some degree of inactivation if the pH of plasma is lowered. Generally, a pH of 4.0-6.0will be useful to alter the viral load effectively; the plasma pH can be lowered without much risk to the patient, as the pH is immediately restored by administering a basic solution.

While the instant invention can be used in any type of viral infection, it is generally admitted by those with the ordinary skill of the art that these are drastic measures of treatment requiring intensive care of the patient when administering the therapy because of the risks involved as the acidosis is induced in a patient. The practice of the invention also requires qualified emergency care physician supervision, a team of support staff to administer the intravenous infusion, and a ready method to test blood samples to determine if the treatment has been successful.

In a preferred embodiment, the instant invention is an effective treatment of all types of viral infections by inactivating the virus through modification of plasma pH to an acidic level that results in significant inactivation of the virus in the body.

In another embodiment, the instant invention is a direct treatment of viral infections by inducing acidosis, the extent of which depends on the nature of the disease and the condition of the patient to tolerate the therapy.

In another embodiment, the instant invention is a treatment of virus infections without regard to the nature of the virus.

In another embodiment, the instant invention involves infusing an acidic solution to a patient to reduce the plasma pH to a predetermined level, maintaining it at the low level for a predetermined time, and then reversing the pH back to normal pH by infusing an alkaline solution, to avoid any side effects of the treatment.

In another embodiment, the instant invention uses acidic solutions to reduce the pH including lactic acid, sulfuric acid, phosphoric acid, hydrochloric acid, carbonic acid, carboxylic acid, carboxylic acid with a ketone group, or a beta keto acid, and sodium bicarbonate to elevate the pH by intravenous infusion.

In another embodiment, the instant invention reduces the pH of plasma to such low levels as a pH of 4, yet momentarily, to inactivate most resistant viruses.

In another embodiment, the instant invention is an effective treatment for patients who are severally ill due to a higher load of viruses that the body is incapable of inactivating, facing death.

In another embodiment, the instant invention treatment is repeated either consecutively or periodically.

What is claimed is:

1. A method of treatment of virus infections comprising:
inducing acidosis of plasma to a predetermined suitable acidic pH by intravenously infusing an acid solution in a patient harboring a clinically significant load of an infectious virus;
maintaining the acidosis for a predetermined suitable time interval;
restoring the pH of plasma by intravenously infusing a basic solution;
repeating steps (a) to (c) consecutively or periodically until a predetermined clinically significant inactivation of the virus is achieved.

2. The method treatment of claim 1, wherein the acid solution comprises lactic acid, sulfuric acid, phosphoric acid, hydrochloric acid, carbonic acid, carboxylic acid, carboxylic acid with a ketone group, a beta keto acid, or a combination thereof.

3. The method of treatment of claim 1, wherein the basic solution comprises sodium bicarbonate.

4. The method of treatment of claim 1, wherein acidic pH is 4.0 to 7.3.

5. The method of treatment of claim 4, wherein the acidic pH is 6.0 to 7.0.

6. The method of treatment of claim 1, where the predetermined time for maintaining the acidosis is 5-60 minutes.

7. The method of treatment of claim 1, wherein the predetermined clinically significant inactivation is 1 to 4 log inactivation in the number of virus particles in plasma or another accessible suitable tissue sample.

8. The method of treatment of claim 1, wherein the virus is an enveloped virus or a non-enveloped virus, or a mixture thereof.

9. The method treatment of claim 8, wherein the enveloped virus is a herpes simplex virus, a poxvirus, a hepadnavirus, an asf virus, a flavivirus, an alphavirus, a togavirus, a coronavirus, a hepatitis D virus, an orthomyxovirus, a paramyxovirus, a rhabdovirus, a bunyavirus, a filovirus, or a retrovirus.

10. The method of treatment of claim 8, where the non-enveloped virus is an adenovirus, a parvovirus, papillomavirus, a polyomavirus, a picornavirus, rotavirus or a calicivirus.
